(19) United States
(12) Patent Application Publication
(10) Pub. No.: US 2014/0288850 A1
(43) Pub. Date: Sep. 25, 2014

(54) SELF-LEARNING OF PLANT GROWTH STRATEGY IN A GREENHOUSE

(71) Applicant: Paskal Technologies Agriculture Cooperative LTD., Ma'alot (IL)

(72) Inventors: Tzvi Avigdor, Nofit (IL); Omri Morag, Kibutz Magal (IL)

(73) Assignee: PASKAL TECHNOLOGIES AGRICULTURE COOPERATIVE LTD., Ma'alot (IL)

(21) Appl. No.: 14/353,702

(22) PCT Filed: Oct. 30, 2012
(86) PCT No.: PCT/IL2012/050427

§ 371 Date: Apr. 23, 2014

Related U.S. Application Data
(60) Provisional application No. 61/553,220, filed on Oct. 30, 2011.
(60) Provisional application No. 61/719,450, filed on Oct. 28, 2012.

(51) Int. Cl. G01N 33/00; G01G 19/00

(52) U.S. Cl. CPC G01N 33/0098; G01G 19/00
USPC 702/19

(57) ABSTRACT

A system for monitoring plants in a greenhouse, the system comprising: a plurality of weighing units, the weighing units distributed in the greenhouse, each of the weighing units is attached to a single plant or group of plants and comprising means for weighing the plant or group of plants, the weighing units are trellising from an elevated wire at one end and connected to the top end of the plant or group of plants at the opposite end; a communication network comprising means for communicating the weight of the plant or group of plants from the weighing units to a central unit; and a central unit, the central unit comprising means for receiving, storing, processing and analyzing data received from the weighing units through the communication network.

### TECHNICAL FIELD

The present invention pertains to a system and method for planning growth strategy in a greenhouse. More particularly, the present invention pertains to investigating the interrelations between growing processes and climate and environmental conditions, growing treatments, and other external effects, in a greenhouse and optimizing growth operation and investment.

### BACKGROUND

The currently leading concept for controlled operation and monitoring of crop growth in a greenhouse uses an already developed model that provides analysis and conclusions on the relation between growth rate and growth conditions, such as climate, environment and cultivation procedures. A growth control system receives indirect indications of plants growth. The data collected are used to construct growth calibration curves, which the model uses as input for analysis, identifying flaws in implementing growth conditions. Indications of plants growth according to this concept may be, for example, leaf thickness, fruit or stem perimeter and plants' weight, which is measured mostly to monitor and control irrigation. Such indications are collected from sensors distributed in a pre-selected area in the greenhouse, the collection of sensors defining a representative station that reflects growth in all other areas, which are not monitored. Thus, instead of constructing a method of operation based on growth data, a predetermined method of operating the greenhouse resets growth conditions according to the data received.

In another way, the greenhouse is divided into areas, where each area has its own deployment of sensors. Each area operates individually, producing growth data that correspond to the growth conditions in that area. Growth conditions should then be corrected for each area separately.

Weighing plants in a greenhouse provides information on their growth rate under known and measurable parameters, e.g., level of temperature, humidity, irrigation scheduling, lighting, fertilizing, ventilation concentration levels of carbon dioxide (CO₂) and others. However, measuring plants weight is not as straightforward as might be appreciated. Non-linear changes and interaction between climate and environment conditions affect plants growth and plants weight measurements. In addition, non-linear, extreme, cultivation activities by the grower or other weight influencing events and parameters should be cleared off in order to monitor and obtain the actual growth rate of the plant expressed by a “clean” growing curve. Leaves removal, fruit picking and trellising process are the main interfering operations that cause changes in plant weight, and are not part of the “clean” growing curve of the plant. Such factors cannot be accounted in the expected gradual response of the plant to its surroundings.

Because of the complexity mentioned above, one type of technique for monitoring plants growth in a greenhouse involves group weight measurement. Such technique provides only calculated average weight of a single plant based on the gross weight of a group of plants from one assumed representing location, as opposed to a plurality of individual plant's actual weight spread all over the greenhouse. Batch size of the plants measured ranges between 5 and 10 meters length of crops. The data gathered allow general detection of growth progress. However, they do not contribute significantly to understanding the connection or correlating between growth rate and different parameters that affect it. This is due to the use of weight sensors that are fixed in place and cannot account for changes of and deviations from normal growth rate resulting from changes in environment or climate conditions in the greenhouse.

One currently used weight measuring device is permanently fixed at pre-selected measuring points in the greenhouse and cannot be relocated or moved to another place. This compounds on monitoring dynamic variations in growth and relating it to seasonal, temporal or local effects. The devices used are composed of two weighing units, one bottom that measures the plant's laying or rooted part, the second trellising that measures the plant's hanging part. The main problem in such weighing method and system is essentially statistical, since they do not provide sufficient information for generalizing global conclusions for a defined period of time, area or the entire volume of the greenhouse. Also they do not encompass deviations from normal growth or explain them properly.

Growth input such as irrigation, fertilization, temperature, lighting, concentration level of carbon dioxide (CO₂) and more are provided to a plant based on data received from sensors distributed in the greenhouse. However, the feedback of the growth results is still gathered mainly under the observing eye of the grower or by manual collection of the harvest data unless they are collected along the growth lines. There is, therefore, a need for collecting data from sensors distributed in a greenhouse that is not mediated by human labor. In every growing system there are variations between plants in different locations in the greenhouse. To date there is no possibility to determine variations in different places of a greenhouse. This also requires that the system and method provide dynamic data collection and analysis means that detect and point to the sources of such variations.

WO 2004/040965 describes an apparatus for measuring and controlling crop growth in cultivation troughs on growth elements such as substrate mats. A cultivation trough is trellising from a first weighing means such as force sensors, while the plant supporting means is trellising from a second weighing means, also force sensors. The load of the plant supporting means can be recorded independently. The second weighing means may be trellising from the first weighing means. The first weighing means measures the full weight of the cultivation trough with crop, plant supporting means and growth element, including water. This ensures that errors in the measured crop weight resulting from, for instance, partial support on the cultivation trough, can be eliminated in further calculations. This apparatus is fixed permanently at a certain location to the construction of a greenhouse, and is immobile. Furthermore, beside detection of water flow in and out of the plant the apparatus does not provide further knowledge on the plants growth behavior.

It is, therefore, an object of the present invention to provide a system and method for monitoring plants growth in a greenhouse that overcome the deficiencies of existing systems and methods.

In yet another object, the present invention provides a novel concept for operating and monitoring crop growth in a greenhouse by extracting a statistical model based on growth, climate, environment, labor and other input collected at the greenhouse and from external sources.

In yet another object, this concept is implemented with a set of tools for constructing the statistical model. The mapping of growth and growth conditions irregularities in a greenhouse is made possible with a modular, movable assembly of multiple weighing units (also referred to as ‘kernel’), communication network for communicating the data collected at the greenhouse and from external data sources, and collecting, processing and analyzing unit that employs an appropriate algorithm for processing and analyzing the input data.

Thus, in yet another object, the present invention provides a system and method for monitoring crop growth using the kernel, which is deployed in the greenhouse in every desirable formation and maps the area it defines. The plurality of weighing units and modularity and mobility of the kernel generate a dynamic picture of the greenhouse with a grid and pixel size, which are set according to the objectives of the system as set by the operator.

Yet another object of the present invention is to provide a decision support system and method for managing a greenhouse that provides insight to the interrelations between plants growth and environmental, external and growing treatment conditions.

Yet another object of the present invention is to provide a system and method implemented in a greenhouse for monitoring plants growth based on measuring and correlating between individual plants weight and different parameters that influence plants growth.

Yet another object of the present invention is to provide a system and method for collecting, processing and analyzing data and predicting growth trends in a greenhouse.

Yet another object of the present invention is to provide a system and method for determining uniformity of plants population in a greenhouse.

Yet another object of the present invention is to provide a system and method for monitoring plants growth in a greenhouse that is dynamic and flexible.

Yet another object of the present invention is to provide a system and method for monitoring plants growth adapted to trellising plants.

Yet another object of the present invention is to provide a system and method for monitoring plants growth adapted for trellising plants circulating in a carrousel trellising system.

Yet another object of the present invention is to provide accurate continuous measurement of plants weight and environmental conditions influencing it.

Yet another object of the present invention is to provide a system and method that optimize plants growth in a greenhouse from an economic perspective.

Yet another object of the present invention is to provide a system and method to evaluate plant growth uniformity in the greenhouse and enable the grower to improve yield.

This and other objects of the invention will become apparent as the description proceeds.

### SUMMARY OF THE INVENTION

In one aspect, the present invention provides a novel approach for operating and monitoring crop growth in a greenhouse by extracting a statistical model from measurement data collected at the greenhouse and from external environment, climate and other sources.

This is implemented in a system and method for receiving information on crop growth from a plurality of weighing units and sensor means distributed in any selected area in a greenhouse. The units and sensors map growth-related parameters such as growth rate in the selected area and irregularities in particular locations, and a communication network transmits the data collected to a processing and analysis unit. Software analysis means correlate between environment, climate and any other growth- and growth-rate affecting conditions, thus providing a method for generating, customizing and optimizing a growth model for a particular greenhouse. This way, the system and method of the present invention provide fast, direct and robust feedback on the data collected and a continuously updated image of crop growth in the greenhouse.

### Kernel

The above is enabled by deploying a moving, modular kernel composed of an assembly of plant weighing units and sensors in any desirable formation over any selected area in the greenhouse. The weighing units are detachable off the plant(s), which allows them to be repositioned at any other desirable spot in the greenhouse. This way the kernel can move around the greenhouse taking a different formation and dynamically redefining a different area for investigating growth. Different areas of the greenhouse are affected differently by environment and climate conditions surrounding it. Therefore, the moving modular kernel provides an optimized solution to dynamically monitor any selected area in the greenhouse and generate a practically real-time, accurate image of crop growth.

Therefore, in one aspect of the invention, the kernel defines an effective spatial zone of plant growth inspected in an effective time unit. The system scans this growth zone within the borders defined for it and produces a set of data. Based on the data collected, the system generates a time-dependent and time-defined picture of the growth zone. The definition of the effective time unit responds to the requirement for extrapolating the data collected over every selected area in the greenhouse or in any other greenhouse. That is, the effective time unit is set to produce a comprehensive image of the growth zone and derive global conclusions regarding the relation between the observed growth and climate, environment and other growth affecting conditions.

In one embodiment, the kernel that comprises a plurality of weighing units provides coverage over essentially the entire greenhouse or a substantial part of it. In one non-limiting example, the distribution is 25 weighing units over one hectare of a greenhouse. It should be noted that any number of weighing units can be distributed over any pre-selected area size in the greenhouse.

The weighing units communicate their readings wirelessly to a central processing unit through a communication network. This assists in their fast and dynamic repositioning and redistribution, providing the real-time picture of the greenhouse with any desirable grid and pixel size.

Lastly, the real-time growth data are transmitted to the data analysis unit, which provides updated image of growth in the entire greenhouse, including spots of deviation from normal growth and growth rate.

To summarize the above, in one embodiment, the feedback system of the invention is composed of three basic elements: (1) weighing units including transducers that convert continuously the measured weight of the plant(s) to electrical signal; particularly, the transducers continuously compensate the measured weight of the plant, which is affected by temperature distortions; (2) a communication network that collects and transmits weighing and environmental data and the individual identification of the weighing unit; (3) data collection unit and processing and analysis software that analyzes the weighing, climate, environment and other growth affecting data and displays a continuous distribution of the growth data in the greenhouse and possible reasons for their occurrence. In this particular embodiment, the continuous data collection of plants weight in a greenhouse, data processing and analysis enables the grower to supervise and monitor growth, detect problematic places, identify variations in growth results and the like.

The following describes the different components of the system, which reduce to practice the general concept of the present invention discussed above.

### System

In one aspect, the present invention pertains to continuous onsite acquiring, processing and analyzing the weight growth of a plurality of individual plants, most particularly tomato plants, relative to climate, environment and other growth- and growth-rate affecting conditions that surround and affect them.

In another aspect of the invention, the greenhouse is perceived as a three dimensional integrative entity with organic connections between its elements and the surroundings and between the elements themselves. Such connections influence the behavior of the greenhouse and affect its productivity. To this end, different parameters that represent such elements or the surroundings are monitored, thus providing a comprehensive and detailed description of the greenhouse vitality.

In one particular embodiment, the system of the present invention identifies problems in the cultivating and supporting systems of a greenhouse and provides indication on problems to be addressed, for example growth uniformity. In one example, irregularities in the irrigation system are identified according to variations in plant weight as a result of irregular water balance cycles. Thus, the system of the present invention essentially operates as a detecting tool that provides indication of malfunctions in the greenhouse.

The history of plants growth determines the normal growth. According to the normal growth values observed, the system calculates the average growth rate and growth deviations detected in the greenhouse. In a third stage, the system receives growth history data, including data related to the environment, and outputs a predicted growth based on growth history. This predicted growth is then compared to the actual growth of an individual plant. The comparison in turn helps tracking the climate, environment and other conditions and the investments (water, fertilizers, ventilation, heating and/or lighting energy, labor etc.) in plant growth that lead to the deviations from the historically determined average growth rate.

In one aspect, the decision support system comprises means for collecting, recording, storing and analyzing plant-related and climate, environmental and any other growth and growth-rate affecting conditions data, weighting and processing the data, providing comprehensive status of the greenhouse based on continuously updated database and optimizing growth from an economic perspective.

In accordance with the above, the present invention provides a system for maximizing profit on the greenhouse. The system performs optimization of plants growth for producing the maximum economic profit that can be achieved from the greenhouse. To this end, costs of investments such as water, fertilizer, humidity, energy (ventilation, heating, lighting, etc.) and labor, plant growth and crop yield are taken into account. The system then produces the optimal value of growth that can be achieved under known conditions. Based on these data, the system generates an optimization curve that maximizes profit as a function of the entire inputs to the greenhouse. In other words, the system is a decision support system that helps concluding what best mode of distributing investments would produce the highest crop yield of a greenhouse. Growing plants in a greenhouse is then essentially quantified to money worth based on the money worth of growth influencing variables.

In another aspect, the present invention provides a global system that unifies accumulated data from one or more greenhouses, and the unified database integrates the experience gathered from different plant growth locations. This allows better understanding of the connection between plants growth and the climate and environmental conditions influencing it. The unified database and the conclusions derived from it project improved insight on how to maximize profit from a specific greenhouse by fine-tuning growth conditions and investments in plant growth such as water, fertilizer, humidity, energy (ventilation, heating, lighting, etc.) and labor.

### Weighing Unit

In one aspect, the present invention provides a feedback weighing system that accurately and continuously measures the weight of individual plants or group of selected plants. The system is adapted to greenhouse trellising systems, in which the plants hang from an elevated gutter wire. A weighing unit with accurate scales is attached to the wire at one end and to the top end of the plant at the opposite end.

In another aspect, the weighing unit employs scales capable of measuring small increments of plant weight. This capability imparts high accuracy to the measuring and analysis system. It essentially resonates with the nature of the feedback system of close monitoring of otherwise undetected events and their impact on plants growth.

In one particular embodiment of the invention, the accuracy of the scales of the weighing unit enables recording increments of ±2 grams for every 10 kg of plants weight.

In another embodiment of the invention, a supervising component in the weighing unit sets the time intervals for taking measurements. In a still particular embodiment, the time intervals are short enough to enable an essentially continuous recording of plant weight and environmental data. In one non-limiting example, the time interval for taking a measurement is between 10 and 30 minutes. However, the time interval can always be set according to the need to detect plants growth response to temporary changes in its surrounding climate and environment. Continuous monitoring of plants weight and climate, environmental and other conditions including labor enables correlating between irregularities such as non-linear weight drops and sudden, local, temporal or seasonal changes in the environment.

In a still more particular embodiment of the invention, a temperature measuring device is placed in the vicinity of the measuring unit.

In still another embodiment of the invention, the temperature measuring device provides high accuracy measurement, in particular an error of ±0.2-0.4° C.

In still another embodiment of the invention, each weighing unit is calibrated by triple or double temperature measurements for each of two measured weights. Calibration is made by measuring different weights with a load cell of a weighing unit at different temperatures. The temperatures are measured with a temperature measuring element in the weighing unit. Then a specific algorithm sets the relation between the measured temperature and the load cell. The calibration essentially correlates the load cell and the weight it experiences with the temperature measuring element and the temperature it measures when weighing a plant. The calibration concentrates on the weight relative to the temperature and disregards the absolute temperature in the surroundings of the weighing unit, measured for example, by the temperature measuring device.

The calibrations procedure is further detailed in the following Method section.

In one embodiment of the invention, the plants in the greenhouse are planted in the ground or in a substrate detached from the ground, such as a trough, but are hanging from an elevated iron or steel wire stretched horizontally a few meters above ground. Weighing the plants is made from above and actually measures the weight of a hanging plant using an intermediating weighing unit. In such procedure, measuring plant weight is accurate and not affected by any interference. In a still particular embodiment of the invention, the method of hanging plants makes use of a hanger having a cartridge with extra wire, hereafter a releasing hanger. The releasing hanger is intended to support the stem that grows beyond the space between the ground and the elevated horizontal wire. In still another embodiment of the invention, the weighing unit forms an integral part with the releasing hanger of the plant.

In still another embodiment of the invention, the weighing unit connects to the releasing hanger in a way that does not disturb the action of lowering the plant during growth. That is, the releasing hanger also provides a functionality of a weighing unit. In still another embodiment of the invention, the weighing unit is mobile, moving together with the hanging wire (releasing hanger) that circulates the trellising plants.

The following details the particular components of the trellising weighing unit (as shown in FIG. 1, element 2 and described further in the description):
(1) Trellising scales (sensor): includes a transducer, which is optionally a dedicated S-shaped scale transducer with a Wheatstone bridge electrical connection and external temperature compensation. Such transducer enables low cost measurement with a very high accuracy.
(2) Optional external temperature sensor, hangs in the vicinity of the plant top end for measuring specific ambient temperature.
(3) Housing: Accommodates sampling and communication means in communication with the scale transducer. In addition, the housing enables an easy, fast and simple connection to the trellising system at its upper end and to the wire (10 in FIG. 2) at its lower end. The housing is able to produce weight and momentum balance along the weighing axis for accurate weight sampling.
(4) Sampling means: located inside the housing and includes, power means, for example a battery based power unit, processing means, for example a microprocessor and signal converting means, for example an ADC transducer, that amplifies the analog signal received from the transducer and samples A/D of weight and temperature. The processing means performs initial data processing from the transducer, for example: background noise filtering, weight event identification, resetting and calibration etc. In addition, the processing means includes specific ID for identifying the weighing unit, allows saving data (data storage) in a local memory and transmitting data to a central unit through the communication unit.
(5) Communication means: located inside the housing and communicates with the sampling means. The communication means communicates between the sampling means, the communication network and the central unit. This communication means includes also network capabilities including data transmission, clock update and configuration and data repeater.
(6) Central unit: manages the communication network and gathers data from all trellising weighing units in the greenhouse. The central unit synchronizes the network and communication with all trellising weighing units. The central unit is fed by a wire power source, characterized in receiving sensitivity as high as possible thereby enabling low power transmission from the trellising weighing units and prolonging working time without replacing the battery for at least 5 years.

As mentioned above, the weighing units may be repositioned in the greenhouse in any desirable location. To this end, they should be conveniently detached from and re-attached to the gutter wire of the trellising system. Therefore, in one particular embodiment the physical structure of the weighing unit is designed to enable relatively quick and easy attachment to and detachment from the gutter wire on the one end and the plant(s) on the other end. The physical structure of the weighing unit is illustrated in FIGS. 11A-E and will be further explained in the section of Detailed Description of the Drawings.

### Data Transmission Network

In one aspect, the system of the present invention comprises wireless communication means for communicating between the weighing units and the data analysis unit. In particular, the weighing units communicate through repeater means distributed in the greenhouse.

The repeater-Repeaters means are distributed in the greenhouse and communicate between the weighing units and an access point, namely the central transmission means. The repeater means also communicate between themselves, thereby channeling wireless transmission between the weighing units and the access point in an indirect way. The indirect transmission through the repeater-repeater means opens communication routes that obviate physical obstacles in the greenhouse that block transmission. For example, the metal or steel structure of the greenhouse may block wireless radio transmission between the weighing units and the access point during movement of the trellising system. The repeater means provide a bypass to which transmission is automatically directed when direct connection with the access point fails. The wireless network defines itself automatically and finds dynamically the possible repeater-repeater route according to the current position of the weighing units relative to the repeater means and the access point relative to each other.

The repeater means communicate with central transmission means of the wireless communication network, and the central transmission means communicates with a central unit, or the weighing units communicate directly with the central transmission means. The central transmission means are located in the greenhouse, and communication between the weighing units and the repeater means or central transmission means enables synchronizing weighing events taken at selected time intervals by the weighing units.

In one embodiment of the invention, each repeater communicates measurement data collected from local sensors in the vicinity of the weighing unit or sensors distributed over the greenhouse to the central unit for data analysis.

In still another embodiment of the invention, data transmission from each weighing unit to the central unit is wireless.

The central unit is connected to the system server through USB communication, and stores updated information that includes also identification, status and measured parameters of every weighing unit and sensor.

In one particular embodiment, identification data of the specific scale unit is transmitted together with the weight data to identify the weighed plant and map its location in the greenhouse.

In one particular embodiment, data transmission is made in a 433 MHz frequency, which is widely used and suitable for short transmission lengths. In still another particular embodiment, data transmission can be made in other frequencies as part of adapting the system to local regulations and approved frequency ranges of different countries. This will increase efficiency of the system and allow better adaptation of the product to the targeted market. As an example, 169 MHz frequency can be used to adapt to the European market. Transmission in this frequency increases range by at least a factor of two, reduces communication problems resulting from scarce use of radio networks in this range relative to 433 MHz, and extends range length by additional increase of transmission power allowed by regulations.

The wireless network is bi-directional. For example, it produces and communicates a fault report and calibration results of operational or dysfunctional weighing units, to the central unit. On the other hand, it enables remote/wireless direct communication for its continuous management and maintenance. Below are some non-limiting examples of the different functionalities of the wireless network:

1. Clock update;
2. Synchronizing the weighing units;
3. Weight calibration of the weighing units;
4. Storing last calibration data;
5. Continuous maintenance and calibration;
6. Unit resetting;
7. Transmission shutdown.

### Processing and Analysis Unit

In one aspect, the system of the present invention comprises a central database and analysis software unit to which the measured weight and environmental conditions are transmitted. This central unit creates and manages a global database of the measured values and runs statistical analysis based on the data collected.

In still another aspect of the invention, the central unit collects, stores and analyzes measurements taken by each weighing unit.

Particular parameters that affect plant growth include but are not limited to irrigation scheduling, drainage, ventilation opening percentage and use of a shadowing screen. In one embodiment, the present invention provides simultaneous monitoring and recording of climate and environmental conditions in the greenhouse and growth rate of the plants. This basically provides accurate correlation between the greenhouse characteristics or functionality and its productivity. More particularly, the growth rate of the plants can be explained by the measured values of climate and environmental conditions and unexpected changes they experience. This actually requires simultaneous recording of, processing and analyzing multi non-linear phenomena. Currently, the interrelations between greenhouse characteristics and functionality and productivity are examined with models of dedicated, experimental farms. The system of the present invention, on the other hand, enables extracting or manufacturing a specific model for a greenhouse in which it is installed, using statistical tools based on the data collected. This system replaces the current methods of experimentally verifying suggested agronomic theories.

In one aspect of the invention, mathematical and statistical calculation of the data gathered provide comparative analysis of actual aggregated plant weight growth relative to predicted individual growth of the plants. Thus, the feedback weighing system provides a spatial mapping of plants growth in a greenhouse and detection of non-uniformities and abnormalities that result from local or temporal irregularities in growth conditions.

Still in another embodiment, the measured values of the environmental conditions are processed according to specially formulated mathematical equations, the equations set the ground for correlating between measured climate and environmental conditions and plants growth.

In still another embodiment, the analyzing step comprises averaging and calculating variations in growth over the entire greenhouse and/or selected area thereof.

The data analysis may further comprise filtering background noise, identifying weighing events and identifying weighing units according to identification data, preferably the identification data comprises spatial location of the weighing units.

Also, the data analysis further comprises providing spatial representation of plants performance and variation of the plants performance with time, the plants performance being expressed in diseases in the plants, growth rate and general response of the plants to development process.

In accordance with the above, the central unit comprises time-domain filters, spatial-domain filters, smoothing filters and edge-enhancement filters. In particular, the time-domain filters and smoothing filters enable smoothing irregularities and highlighting non-linear events associated with the growth rate of plants. The irregularities comprise picking, deleafing, trimming and lowering, and events that comprise variations in climate and environmental conditions in the greenhouse.

The spatial-domain filters, edge-enhancement filters and smoothing filters enable, in one particular implementation, smoothing and highlighting location-dependent phenomena and defining areas boundaries in the greenhouse according to the phenomena. The location-dependent phenomena comprise local development of plant performance expressed in diseases in the plants, growth rate and general response of the plants, working-boundaries of a worker, areas of non-uniform circulation of humidity, variations in temperature, distribution of pesticides and non-uniformities in plants fertilization.

With regard to the above filters, the greenhouse may further comprise means for correlating between present growth rate and past values of growth-influencing parameters. Such means enable a correlation lacking any in-advance information of the phenomena generating values of the growth-influencing parameters.

Still in another embodiment, the central data analysis unit comprises statistical means for calculating growth rate data in plants in the greenhouse. Such means enable producing and optimizing economic and cost-effective models of growth of plants in the greenhouse based on accumulated, historical and statistical data and quantifying growth strategy for analysis and decision-making activities. In particular, the growth strategy comprises optimizing plants growth-rate influencing conditions. The growth-rate influencing conditions comprise fertilization, irrigation and water balance, heating, carbon dioxide (CO₂) concentration levels, application of ventilation, screening, plant treatment regimens and labor performance of any particular worker in the greenhouse. Specifically, the plant treatment regimens comprise plants lowering, deleafing, trimming, picking, harvesting and spraying. The labor performance relates to the working quality of a worker in a particular area in the greenhouse and its influence on growth rate and quality of crops.

As detailed above, the growth strategy enables a grower to maximize profits from the greenhouse by adjusting the investments in the different parameters that affect plants growth to achieve the best profitable result.

In still another particular embodiment of the present invention, the following software architecture is implemented in the data collection and analysis unit as follows:

The architecture of the software system is composed of the following three basic parts:
1. CDB—Central Data Base—is used for data storage and transmission between the different components of the system. In a particular application of the present invention the CDB operates on online global platform for data management in a greenhouse and generates a comprehensive database of the different data of the greenhouse. Interfacing with this system creates a platform on which statistical analysis of greenhouse data are carried out. The CDB also serves as a comparative tool between yields of different greenhouses and for intercepting problems.
   In a further advanced application, an independent data assembling system is used. Such system employs communication interface for data assembly using software dedicated for managing communication. It is further contemplated that communication interface management software systems leading in the field are incorporated into the CDB of the present invention.
2. Data collection system—in charge for collecting data from end units and from external resources such as Climate & Irrigation controller, labor registration system and more. In particular, the data collection system performs assembling information from the central unit that supervises the weighing units. The central unit gathers weight, temperature and other environmental data from the end units transmitted to the database. The system also transmits data to the end units. Particular examples are calibration, gross weight and configuration. The system also synchronizes between the units and supervises the radio network. The system assures correct and efficient data collection for the DSS (Decision Support System) for preserving high performances.
3. DSS—Decision Support System—is self-learning, supports decision making, aids the grower, and provides tools for improving growing strategies in the greenhouse. The system supports collecting, analyzing and displaying data and managing and examining crops growing strategies.

In a still particular embodiment of the invention, the system supports the following modules:
(a) Filtering and signal processing: This module collects, identifies and processes the data collected for display or analysis performed by other modules. The filters enable proper filtering of the information but prevent loss of important data critical for performance analysis.
   Another role of the module is in reducing the quantity of information collected by operations such as discarding superfluous data and use in independent functions for modeling information.
(b) Correlation and connection analysis: This module identifies factors and produces cause and effect connections, for example between present growth rate and previously recorded environmental data, using statistical tools—a result between grower cultivation actions under different growing strategies and actual growth results. The statistical models allow quantifying growth strategy for analysis and decision-making activities by the grower. Correction of this kind is based on advanced mathematical principles and mapping relevant connections between the greenhouse growth parameters. Defining these connections makes use of historical/prior data of previous crops and data received from prototypes of the system. The system examines the strength of the factors found and improves itself with time and additional accumulated experience in monitoring the greenhouse.
(c) Growth simulation module—This module enables finding crop growth feature and creates statistical function of different data accumulated by the system. Running computer simulations then follows, intended to predict behavior and growth products. This is in order to find best growth strategies disposing the need of specific preliminary experiment in the greenhouse. The system is self-learning, preserving the data received from the scale units and weighting them along with CDB when examining the simulations. This functionality updates perspective based on a large accumulated database and further educates the grower.
(d) Analysis and user interface: This module is central to the system, since it enables the grower to optimize controlled parameters in the greenhouse: fertilization, irrigation/water balance, heating, carbon dioxide (CO₂) concentration levels, application of ventilation, screening, plant treatment regimens (e.g., lowering, trimming, deleafing, harvesting and picking) in order to maximize profit. At a first stage, the software presents the grower with the working point and factors influencing growth yield correlated with the different parameters. Presentation is designed to be convenient to growers inexperienced with operating sophisticated software of this kind. For this reason the interface that facilitates access to the grower is of great importance. The grower is able to make a decision based on facts, experimental results and accumulated knowledge.
   The present invention also contemplates on a further advanced module that enables relating accumulated knowledge to economic target function. The software is based on analysis of accumulated knowledge using newly developed algorithms
(e) Alarm system: This module serves as the interface alarm for the grower, such as warning on irrigation problems, abnormal growth rate, plants diseases etc. Alarming is made according to the type of problem and addressee using relevant communication means, SMS, e-mails etc.
(f) Reports and data display generator: The role of this model is to present different data of the information accumulated in the system. For example, daily, monthly, annual or seasonal growth rate display of the greenhouse and location of the weighing units, problems and malfunction display, costs etc. This module supports a wide range of displays: graphs, clocks etc.
(g) Connection to external databases: This module enables the grower to communicate with external systems, such as other greenhouses owned by him or other growers. This inter-communication enables learning and comparing beyond a single greenhouse. It is also contemplated according to the concept of the present invention that this module interfaces with online platform, thus allowing built-in inter-connection between multiple greenhouses.
(h) Internet connection and external user interface: A secure communication network enables the grower remote supervision and inspection of the different greenhouse data, remote operation of the system, import of relevant data such as costs of stock for analysis and comparison etc. Such interfacing essentially provides freedom of operation to the grower and immediate response in emergency situations, keeping an updated constant alert.

Statistical analysis of the data received then follows, correlating between measured growth of the plants and the greenhouse environmental conditions, global and local. Several of the processing operations carried out in the database central unit and which are necessary to establish growth pattern are detailed below:
(1) Filtering and neutralizing temporary weight changes: This screens out non-linear or sudden growth changes in the plants. Such changes are attributed to irregular, probably local, environmental effects or to agricultural or cultivating activities taken by the grower. The data remained can then be analyzed in order to establish a normal pattern of growth of the plants.
   FIG. 6 that will be discussed further in the description, displays graph representation of the different stages of data processing, starting from the raw data and ending with the calculated growth. As can be seen, the raw data graph shows a drastic fall in plants weight between 15:00 and 16:00 hours, essentially reflecting a non-linear event. Examples of such events may be trimming, lowering, deleafing, harvesting, picking and any other agricultural cultivating activity that reduces plant weight. These data should be filtered out to enable monitoring, supervising and understanding of the gradual plants growth. The functionality of the central processing and analysis unit is particularly designed to filter these data out, thereby receiving a correct picture of growth rate.
   In another alternative embodiment, non-linear affecting events are already embedded in the database of the central unit and are called upon when detecting irregularities such as the one shown in FIG. 6.
(2) Orthogonal functions generating graph representation of daily aggregated plants weight: Predetermined mathematical functions, especially formulated for the purpose of monitoring greenhouse plants growth, operate on the data assembled at the database. These functions are orthogonal to each other, meaning each of them features an independent aspect of the greenhouse growth environment. The behavior of these functions over the hours of a day (as shown in FIG. 5) is then used to generate growth rate graph of the plant (explanation to follow), in particular a daily growth rate. A combination of the coefficients of the functions with a certain linear function provides the basis for constructing the mathematical representation of all daily weight growth curves.
   FIG. 6, partly discussed above, includes also mathematical graph display of daily aggregated growth based on five orthogonal vectors perpendicular to each other in a five dimensions field. These vectors are essentially a product of the mathematical and statistical functions operating on particular values of the environmental variables of the greenhouse.
(3) Multi-variable least squares regression analysis describing aggregated daily growth: The data collected from the plurality of weighing units at each given measurement time are aggregated and manipulated in linear least squares regression analysis. This produces a line of development of average growth of a single plant in the greenhouse. The curve of average growth of a single plant weight is then compared with the mathematical representation curve based on the orthogonal functions.
(4) Analysis and 3-D display of spatial vectors: The greenhouse is essentially a 3-D entity influenced by multi-dimension parameters. For example, radiation from the sun affects locally on different locations in the greenhouse. It is, therefore, reasonable to correlate between growth rate of plants and their particular locations all across the greenhouse. Thus the characteristic of spatial growth and growth rate is obtained.
(5) Analysis and 3-D display of daily growth rate as a function of two variables: Two different variables are elected for analyzing their combined effect on growth rate of plants in the greenhouse. A particular example taking radiation and temperature as two prominent variables is presented in FIG. 10 and discussed in the following description.
(6) Optimization of controlled variables in the greenhouse: An advanced functionality of the central unit essentially produces feedback and recommendations to the grower for improving growth by tuning environmental growth conditions. This is based on the data collected from the weighing units and sensors and further knowledge accumulated from manipulating these data.

### Method

In one aspect, the present invention provides a method of acquiring information on and insight to the relation between plant treatment (irrigation, fertilization, trellising, deleafing, harvesting, timing within 24 hours or growth cycle, labor etc.) and plant growth processes. To this end, the method of the present invention isolates outstanding values of meaningful or significant parameters using appropriate data analysis means and enables monitoring and controlling growth in a greenhouse. For example, detection of irregular or exceptional values is made possible by the method of the present invention using the following techniques:
(a) Weighing plants, measuring and collecting climate and environmental conditions at short intervals of time;
(b) Monitoring plants weight, growth rate and climate and environmental conditions over a substantial period of time;
(c) Calculating and predicting growth rate based on mathematical and statistical models and providing explanations for spatial variations in growth over the entire greenhouse and/or selected area of it.

In one aspect, the method for monitoring plants in a greenhouse comprises:
(a) measuring the weight of a plant or a group of plants from a top end of the plant or group of plants with a plurality of weighing units distributed in a greenhouse and trellising from an elevated wire between the wire and plants;
(b) measuring values of climate and environmental conditions surrounding the plant or group of plants;
(c) importing climate, environmental and any other growth-affecting conditions data surrounding the greenhouse;
(d) recording the weight values measured in the weighing units;
(e) recording the values of climate and environmental conditions;
(f) transmitting the weight and climate and environmental values to a central unit through a communication network; and
(g) storing, processing and analyzing the weight and climate, environmental and any other growth-affecting values at the central unit.

Still in another embodiment, the method further comprises calibrating scales of the weighing unit according to temperature. In general, the calibration of the scales comprises:
(a) measuring first, second and third weights with the scales at a selected first temperature;
(b) measuring first, second and third weights measured with the scales at a selected second temperature;
(c) generating a calibration curve based on the weights measured at first and second temperature.

The following table further details the steps of the calibration procedure for calibrating the scales of a weighing unit:

### Magnet Calibration Procedure

| | Action | Result | Wait For | Go To |
|---|---|---|---|---|
| 1. | Place magnet | Start magnet | LED flash 1 | 2 |
| | | calibration | | |
| 2. | Verify no | Tare | LED flash 2 | 3 or 11 |
| | weight | | | |
| 3. | Place | Gain | LED flash off 1 | 4 or 11 |
| | reference | | | |
| | weight | | | |
| 4. | Remove | | | 5 or 11 |
| | reference | | | |
| | weight | | | |
| 5. | Place weight1 | Verify weight1 | LED flash off 2 | 6 or 11 |
| 6. | Remove weight1 | | | 7 or 11 |
| 7. | Place weight2 | Verify weight2 | LED flash off 3 | 8 or 11 |
| 8. | Remove weight2 | | | 9 or 11 |
| 9. | Place weight3 | Verify weight3 | LED flash off 4 | 10 or 11 |
| 10. | Remove weight2 | | | 11 |
| 11. | Remove magnet | End magnet | | |
| | | calibration | | |

In particular, the calibration curve is specific for each scale and independent of absolute temperature in the surrounding of the weighing unit.

In one embodiment, the method of the present invention further comprises monitoring climate and environmental conditions in the greenhouse in the vicinity of plants weighed by the weighing units or at selected places in the greenhouse.

In another aspect, the present invention provides a method of constructing a decision support system for a greenhouse, the method comprising:
(a) distributing a plurality of weighing units in the greenhouse, where the distribution comprises attaching the top end of each weighing unit to an elevated wire, where the elevated wire hangs above the floor of the greenhouse in a trellising system, and attaching the lower end of each weighing unit to the top end of a plant or a group of plants;
(b) setting a central unit that comprises functionalities of receiving, storing, processing and analyzing data received from the weighing units;
(c) setting a wireless communication network that communicates between the central unit and the weighing units and between the weighing units and central communication means and repeater means distributed in the greenhouse. The repeater means communicate between the weighing units and the central transmission means that communicates with a central unit, or the weighing units communicate directly with the central transmission means;
(d) distributing a plurality of climate and environmental sensors in the greenhouse, where the sensors communicate values of measured parameters to the central unit through the communication network;
(e) importing climate, environment, irrigation, labor and other growth- and growth-rate affecting registration data from external sources; and
(f) synchronizing weighing events taken at selected time intervals by the weighing units.

In one particular embodiment, the climate and environmental sensors are temperature sensors placed in the vicinity of the weighing units.

In still another particular embodiment, the climate and environmental sensors are selected from temperature sensors, air and soil humidity sensors, radiation sensors, carbon dioxide concentration level sensors, water and drainage status sensors and radiation sensors.

All of these sensors may be connected with a wire to the weighing unit and transmit their readings through a wire with a sufficient length to allow them to stay in their places.

In one embodiment, the present invention provides an integrated system that manages the database of one greenhouse or more. Such system integrates the databases of the greenhouse(s) with each other, compares growth and climate and environmental data collected in each greenhouse, performs comparative study of growth in all greenhouses and derives conclusions regarding the parameters that influence growth. The integrated system essentially provides improved insight and understanding of plants growth in a greenhouse in general by assembling experience gathered from different sources and at different locations. The database is, therefore, richer in data, which further enables optimizing growth of plants from an economic perspective. Thus, better cost-effective plans for plants growth can be customized according to the particular characteristics of each greenhouse.

### BRIEF DESCRIPTION OF THE DRAWINGS

FIG. 1 is a schematic drawing of a network weighing system overlaid in a greenhouse.

FIG. 2 is a zoom-in view of a weighing unit trellising from a hanging wire in a greenhouse.

FIG. 3 is a schematic illustration of the greenhouse plant weight monitoring, data collection and analysis system and handling communication using a network cloud.

FIG. 4 is a block diagram of the functionalities of the monitoring, data collection and analysis system.

FIG. 5 is a graph display exemplifying mathematical and statistical analysis run over the collected data.

FIG. 6 is a graph display of aggregated and calculated daily growth of the plants in a greenhouse before and after filtering non-linear variations in plants weight.

FIG. 7 is a graph display of the aggregated and calculated daily growth of the plants in a greenhouse after filtering non-linear variations in plants weight.

FIG. 8 is a 3-D graph display of greenhouse space impact on variations in plants weight.

FIG. 9 is a 3-D graph display of normalized spatial variation of the weight of greenhouse plants.

FIG. 10 is a 3-D graph displaying aggregated daily weight of plants as a function of two variables.

FIGS. 11A-E illustrate a physical structure of the weighing unit.

### DETAILED DESCRIPTION OF THE DRAWINGS

The following is a detailed description of the specific illustrations presented in the drawings and particular embodiments of the present invention.

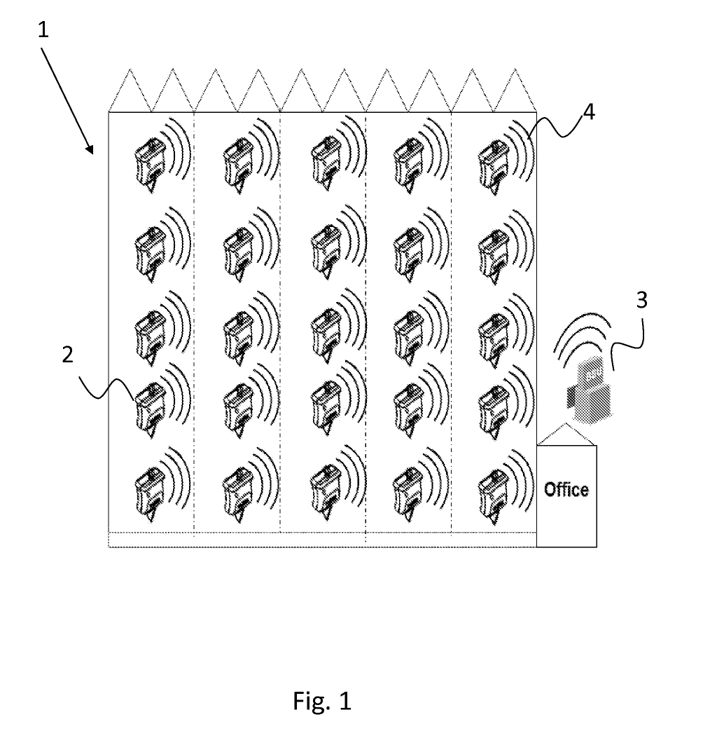

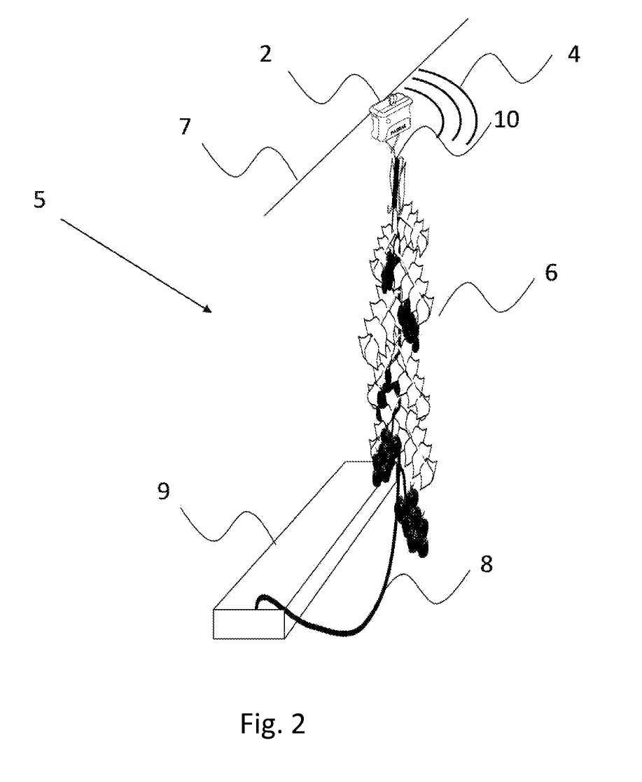

FIG. 1 schematically illustrates a configuration of the network weighing system (1) spread over a greenhouse. This system comprises a plurality of weighing units, each unit (2) attached to an individual plant (6, shown in FIG. 2 ahead). The distribution of multiple weighing units provides efficient spatial coverage of the greenhouse that enables forming a comprehensive picture of plants growth. Coordinated monitoring of multiple plants accompanied by simultaneous recording of environmental conditions provides an accurate description of the greenhouse vitality status at any given moment. The distribution of multiple weighing units (2) all over the greenhouse sees it as an organic integrative entity, of which each location in it provides relevant information on its operation.

Each unit (2) communicates wirelessly (4), directly or indirectly using repeater means, with a central unit (3) for collecting data, termed in the drawing as the “office”. The following describes the particular components of a non-limiting embodiment of a weighing unit (2), the central data collection unit (3) and means for communicating between them.

As can be seen in FIG. 2, illustrating the zoom-in view (5) of a single unit (2), weighing unit (2) suspends from an elevated wire (7) and connects between the wire (7) and the metal hook (10). The metal hook (10) actually hangs from the weighing unit (2) instead of hanging directly from the elevated wire (7). This way the weighing unit (2) can move with the plant (6) along the wire (7). This dedicated configuration of a weighing unit (2) to an individual plant (6) is particularly adapted to “carrousel” trellising systems used in a greenhouse. Water balance of the plant (6) is made through a base (9) to which the lower part of the plant's stem (8) is connected. The configuration of connection between the weighing unit (2) and the plant (6) provides close monitoring through its entire life span and allows deriving more advanced conclusions on plants growth in the greenhouse in view of constant or changing environmental conditions.

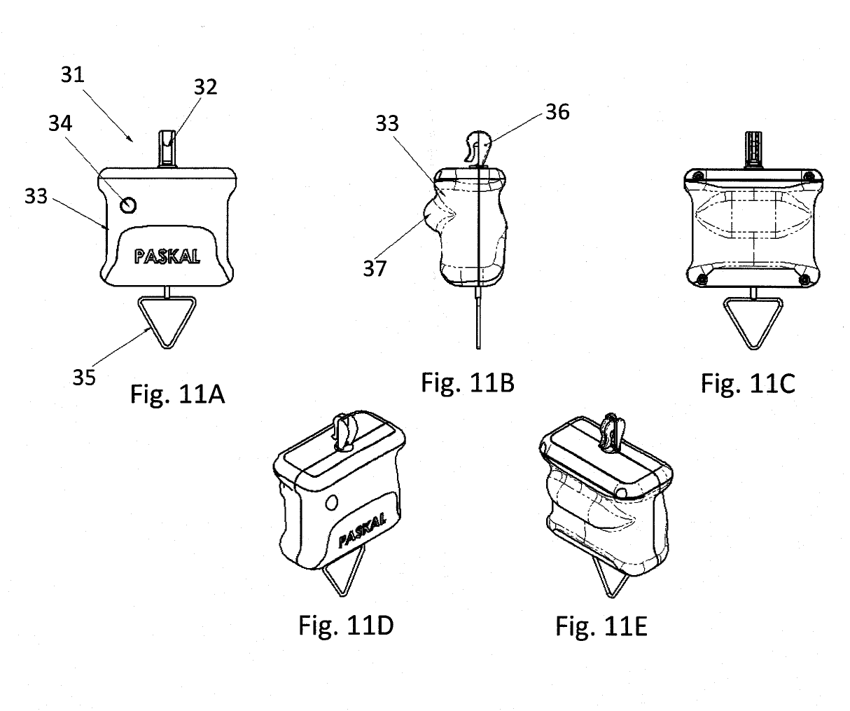

FIGS. 11A-E illustrate different perspectives of the physical structure of the weighing unit (31). FIG. 11B is side view of the weighing unit showing the upper anchor (36) for attaching to an elevated trellising gutter wire (not shown). As seen, the anchor (36) has a shape of a hook with a narrow opening, which allows attaching the anchor (36) to the gutter wire without a risk of unintentional detachment. On the other hand, the opening is wide enough to conveniently detach the weighing unit (31) from the wire without applying a significant pulling force. FIG. 11A shows that the anchor (36) has a round contact (32) that connects to the trellising wire allows tendency of the weighing unit towered the plant angel thus, eliminates side forces on the (31). The anchor (36) is attached to the housing (33), and preferably forms an integral part of it, for example in a single cast or mold. A lower upside down triangle (35) is attached to the housing (33) allowing safe and easy insertion of a standard metal hook (not shown) connected to the upper end of the plant(s). This enables firm grip of the plant(s) without slowing down the lowering process as the plant(s) grow. The triangle (35) is preferably made of a strong material that can bear the heavy weight of the plant(s), e.g., metal, metal alloy, steel and the like. A magnet (34) is located inside a groove in the housing (33). The magnet (34) enables external manual operation of the weighing unit (31), resetting of the scales inside, manual data transmission and manual calibration without opening the housing (33). As shown in FIG. 11B the housing (33) is designed ergonomically, having a round bulge (37) at its upper portion, which allows safely holding, lifting and attaching/detaching the weighing unit (31) without shaking it or the trellising gutter wire. FIGS. 11C-11E show further back and front and back perspective views of the physical structure of the weighing unit including its components discussed above.

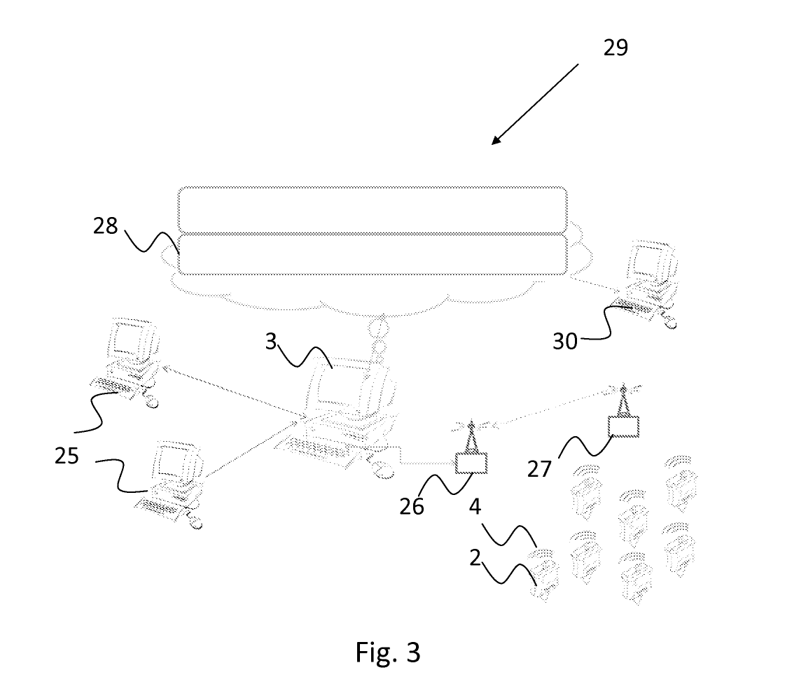

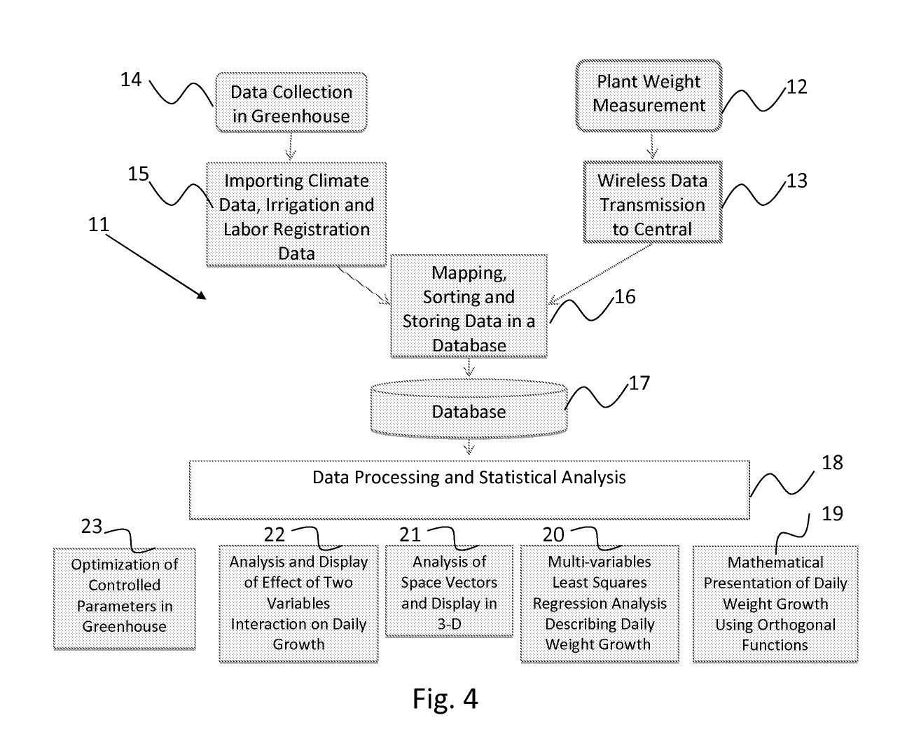

FIG. 3 illustrates a particular configuration (29) of the invention, using internet communication through the services of internet cloud (28). Beside web services, the connection through the internet cloud (28) provides user interface for developing growing strategy support, data mining and processing and LIDAM algorithms. The service of the internet cloud (28) provides global networking with remote databases from which information that is relevant to plant growth in the greenhouse can be retrieved. For example, a remote database (25) transmits climate and irrigation data and labor and crop registration to the servers of the internet cloud (28). The central unit (3) or a remote user (30) can then access these servers and retrieve the information it needs for completing the climate and environmental picture of the greenhouse. This in turn is integrated with the data assembled locally at the greenhouse and facilitates processing, analysis and decision-making regarding plant growth in the specific greenhouse. The internet cloud (28) also enables the central unit (3) itself to share its database with remote databases and data processors or allow remote supervising and control over the greenhouse. For example, the central unit (3) can send continuously an updated status report to the servers of the internet cloud (28) that can then be retrieved by the grower or supervisor using internet communication means. The database in the central unit (3) can also be used by central units of other greenhouses or retrieved by remote processors for comparative or for further analysis.

FIG. 3 features another embodiment of the invention, illustrating central transmission means, also named access point (26) and repeater (27), which are wireless communication means located in the greenhouse and mediating communication and data transmission between the weighing units (2) and the central unit (3). The weighing units (2) move together with the moving trellising system and change their location relative to the access point (26). Transmission between the weighing units (2) and the access point (26) may then break if blocked, for example, by objects such as the metallic structure of the greenhouse. The repeater (27) obviates such obstacles, enabling wireless communication with the weighing units (2) from every angle at the greenhouse and mediates the data transmitted to the access point (26). The access point (26) is in constant communication with the central unit (3) and transmits that data received from the weighing units (2) through the repeater (26).

Particular implementations and examples of the functionalities discussed above are displayed in graphs in the following Figures.

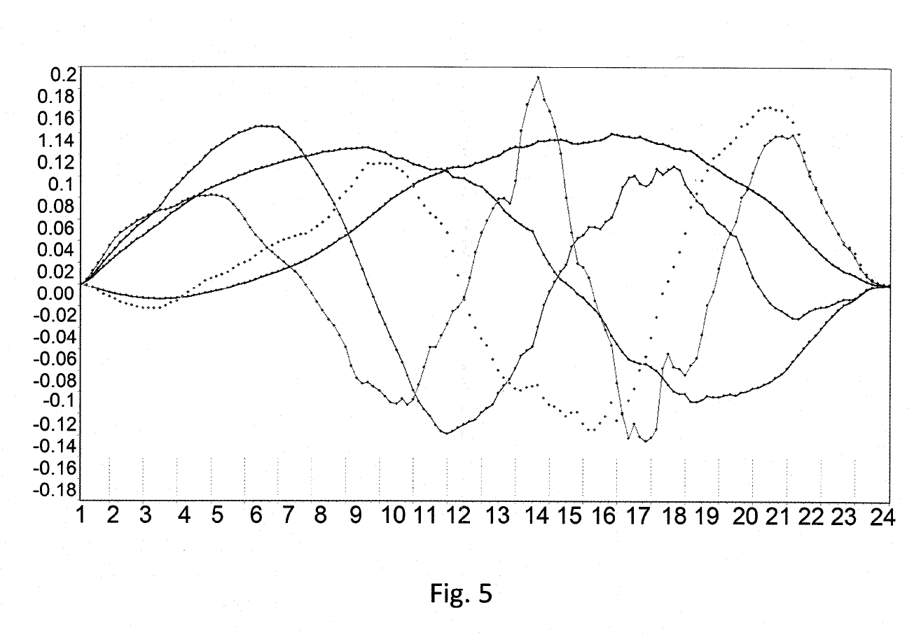

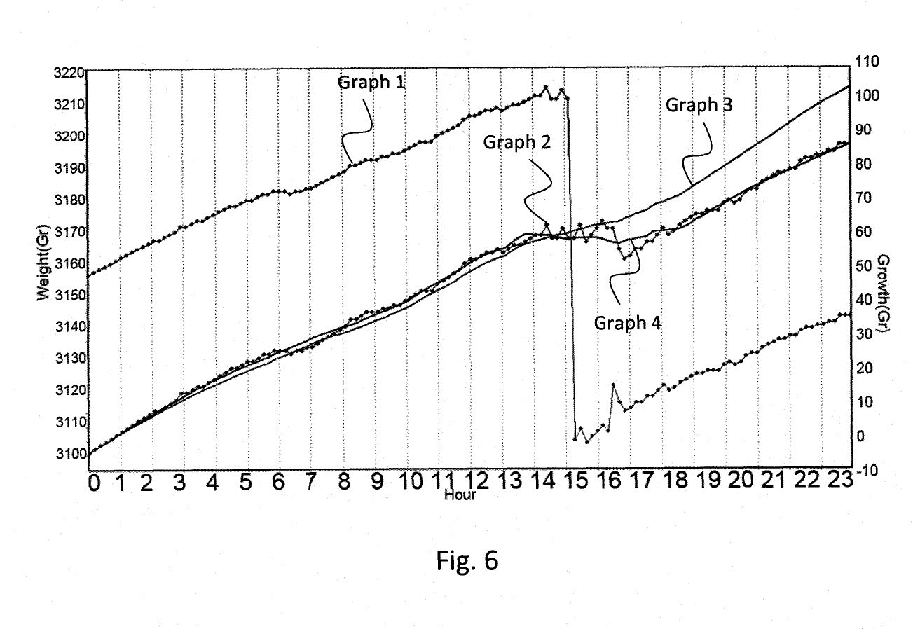

FIG. 5 displays time-dependent behavior of five predetermined orthogonal functions that form the basis of processing and analysis carried out in the central unit. As input, these functions receive the environmental data of the greenhouse. The data are then manipulated according to specially formulated mathematical equations, which set the ground for correlating between the environmental conditions and plants growth. FIGS. 6-10 actually display the second level of processing and analysis based on these five orthogonal functions.

FIG. 6, as partly discussed above, displays the different stages of the processed data in the central unit. Graph 1 records the raw aggregated weight of all measured plants. As can be seen, gradual growth of the plants is detected in the first hours until afternoon. This is an obvious indication of normal behavior responding to environmental conditions supplied to the plants in the greenhouse. At about 15:00 hours a drastic weight drop occurs that can only be attributed to a type of interruption unpredictable by the linear growth of the plants. As mentioned above, the drastic weight drop can result from agricultural cultivating activities or diseases that attack the plants and cause weight loss. Graph 2 is a suitable presentation of the gradual rise of aggregated weight with time after filtering different interruptions influencing weight. Graph 3 is based on the five orthogonal functions. Its basic role is to provide a predicted behavior of weight increase relative to the calculated growth. Graph 3 serves in fact as a reference to average weight increase calculated based on the accumulated weight measurements of the plants. Graph 4 represents the calculated average weight of the plants measured at each point in time during the day. Averaging is made by least squares regression analysis after filtering non-linear interruptions such as the drastic weight drop experienced in Graph 1. The gradual growth of average weight exemplifies normal behavior responding to the efficacy of the environmental conditions in growing plants. Comparing between calculated average weight of an individual plant, Graph 4, and predicted aggregated weight, Graph 3, provides insight on environmental disturbances that deviate plants growth. Deviation can be positive as seen in FIG. 6 or negative, leading the grower to relate it to environmental factors, local, temporal, seasonal or others, that caused it. When such factors are located, corrective measures can than be taken to handle them in order to optimize growth and growth rate.

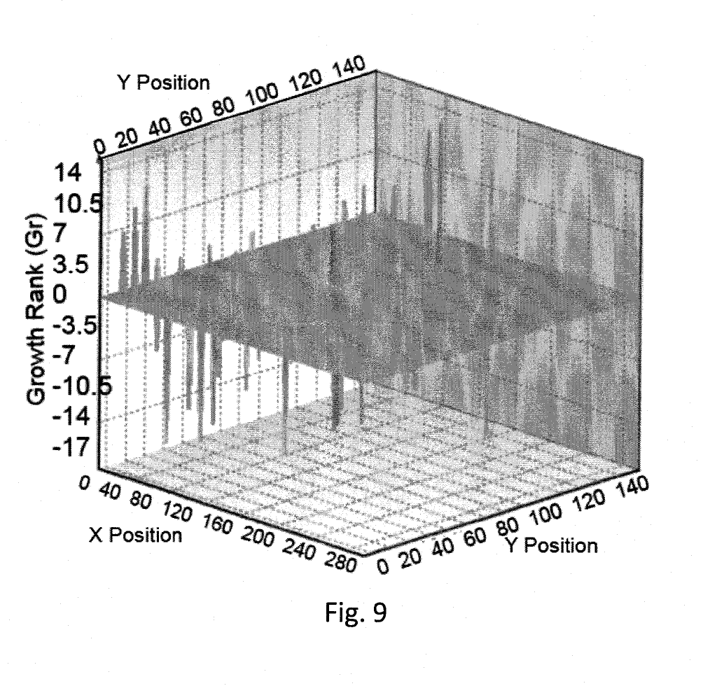

FIG. 9 displays a 3-D graph representation of local factors influencing growth rate and plants weight. A selected area of the greenhouse is mapped and displayed using arbitrary spatial {X,Y} coordinates. Hot spots of sharp deviations from the normal growth are detected after normalizing the data to a zero base-plane. The graph clearly shows the occurrence of positive and negative weight variations at particular places in the greenhouse or a selected area of it. Such weight variations exemplify the irregular spatial variations in plants growth that contribute to the difference between the weight of a single plant and the predicted one as discussed above.

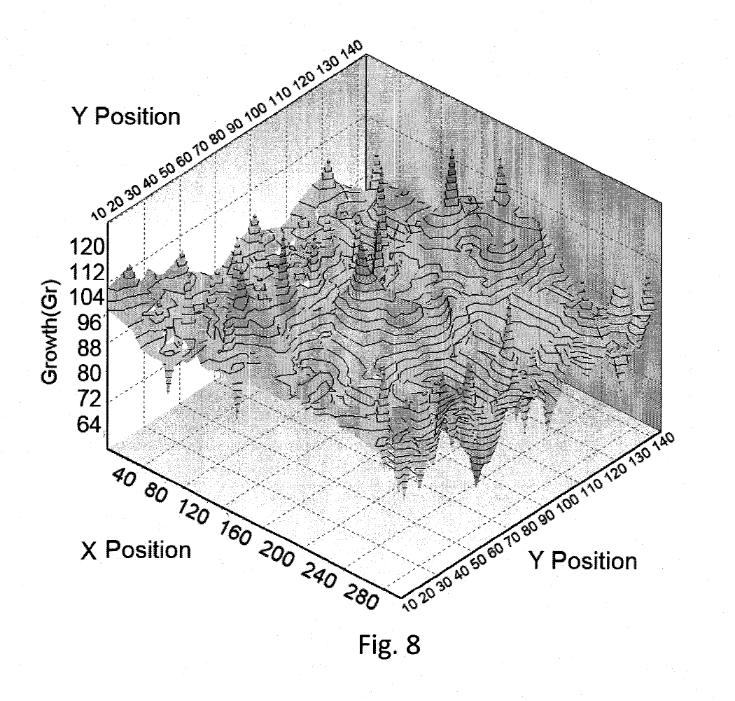

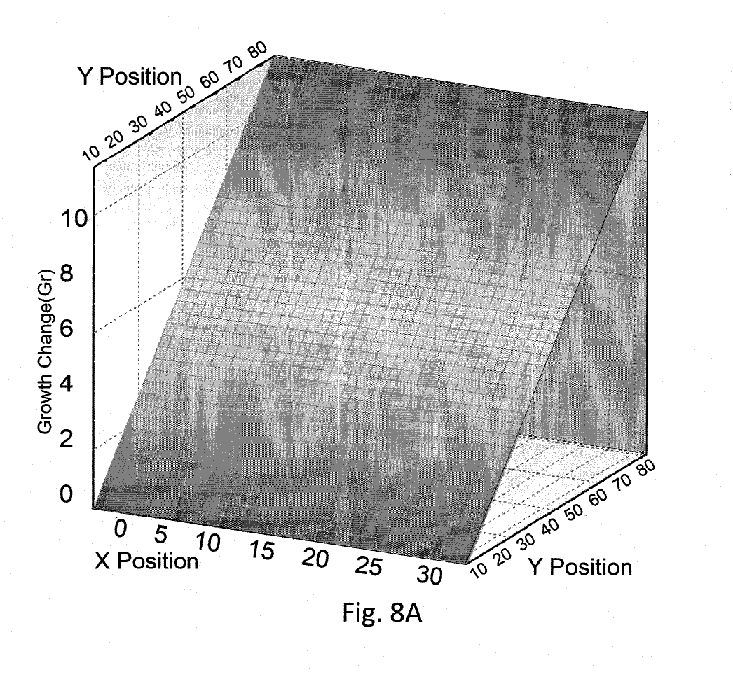

FIG. 8A displays a basic plan of spatial distribution of plants weight in a greenhouse or a selected area of it. Again here, a selected area of the greenhouse is mapped and displayed with arbitrary {X,Y} coordinates. The growth change is measured and converted to arbitrary units. The rise of this change when progressing with the Y coordinate reflects the influence of certain environmental factors that come into play. Such factors could be decreased or increased exposure to radiation, enhanced humidity and temperature, concentration level of CO₂ and so on. FIG. 8 essentially exemplifies the monitoring of spatial variation of growth of plants in a greenhouse by the system of the present invention.

FIGS. 8A and 9 display spatial analysis over the greenhouse layout. FIG. 8A shows significant growth decrease along the greenhouse length (rows). FIG. 9 displays significant deviation from the average of specific plant over the greenhouse layout.

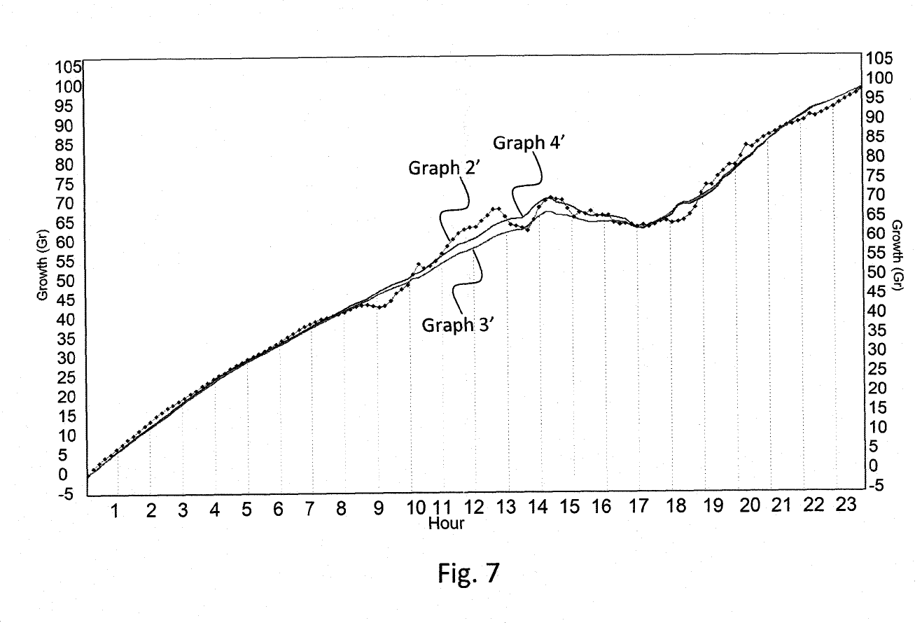

FIG. 7 is another example of calculated average weight of individual plant compared with the predicted one. The predicted aggregated weight is based on manipulation of input data by the five orthogonal functions. A very high correlation between the calculated average and predicted weight is found in this measurement. The raw data, after filtering non-linear effects, also follow the same pattern of growth. These results actually indicate that the environmental conditions are highly uniform over the greenhouse space, and the plants responded similarly at different locations in the greenhouse.

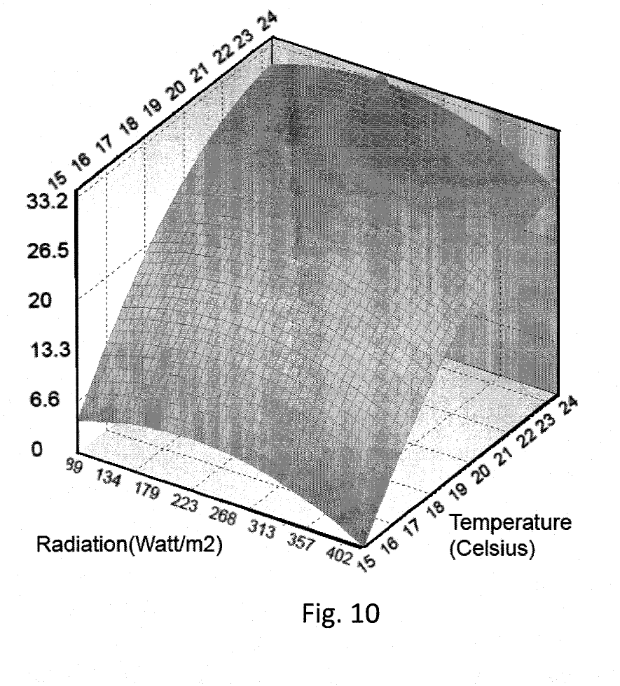

The graphs displayed in the previous Figures enable drawing conclusions regarding the influence of environmental conditions. However, the direct connection between such conditions and growth of the plants has not been presented. FIG. 10 provides such picture, in which plants growth, converted to arbitrary units, responds to the co-effect of temperature and radiation. As can be seen, increase of radiation above a certain level imparts negative effect on plants growth to the point where no or diminished growth takes place. Plants growth increases as temperature decreases at any given level of radiation. The combined impact of the two environmental variables can be used to tune the conditions for a desirable growth rate.

Although selected embodiments of the present invention have been shown and described, it is to be understood the present invention is not limited to the described embodiments. Instead, it is to be appreciated that changes may be made to these embodiments without departing from the principles and spirit of the invention, the scope of which is defined by the claims and the equivalents thereof.

1-48. (canceled)

49. A system for monitoring plants in a greenhouse, the system comprising:
a plurality of weighing units, the weighing units distributed in the greenhouse, each of the weighing units is attached to a single plant or group of plants and comprising means for weighing the plant or group of plants, the weighing units are trellising from an elevated wire at one end and connected to the top end of the plant or group of plants at the opposite end;
a communication network comprising means for communicating the weight of the plant or group of plants from the weighing units to a central unit; and
a central unit, the central unit comprising means for receiving, storing, processing and analyzing data received from the weighing units through the communication network.

50. The system of claim 49, wherein the weighing unit comprises a housing, the housing comprising connecting sampling and communication electronic means to a scales transducer; scales for measuring weight of the plant or group of plants, the scales comprising a transducer and temperature measurement means for calibrating measured weight; sampling means for transforming A/D signaling of the weight and temperature measurements; and communication means for communicating with the sampling means and between the sampling means and the communication network, wherein the sampling means comprising means for filtering background noise, identifying weighing events, resetting and calibrating, and memory means for storing data, the data comprising plant measured weight and identification data for identifying the weighing unit, preferably the identification data comprising spatial location of the weighing unit, preferably said calibrating is made according to calibration tables stored in the memory of said system.

51. The system of claim 50, wherein said housing of the weighing unit comprises an anchor attached to the upper face of said housing for detachably connecting or disconnecting to said elevated wire, a round contact on said anchor, said round contact connecting to the elevated wire and eliminating side forces from shaking said weighing unit, an upside down triangle for connecting or disconnecting to a hook attached to an upper end of a trellising plant or group of plants, a magnet located inside a groove in said housing for external manual operation of said weighing unit, resetting of scales inside said weighing unit, manual data transmission and manual calibration without opening said housing, said housing is designed ergonomically for safely holding, lifting and attaching/detaching said weighing unit without shaking it or the elevated wire.

52. The system of claim 49, wherein the communication network communicates with the weighing units and with the central unit wirelessly, wherein the communication network comprises repeater-repeater means distributed in the greenhouse and central transmission means located in the greenhouse, the weighing units communicating wirelessly and continuously monitoring communication with the repeater-repeater means, the weighing units and communicating directly with the central transmission means upon failure of communication with the repeater-repeater means, the communication between the weighing units and repeater-repeater means being re-routed according to base signal transmitted from the central transmission means to the repeater-repeater means, the communication between the weighing units and the repeater-repeater means enables synchronizing weighing events taken at selected time intervals by the weighing units.

53. The system of claim 49, further comprising means for monitoring environmental conditions in the greenhouse, the environmental conditions are monitored in the vicinity of the weighing units or at selected locations in the greenhouse, wherein the means for monitoring environmental conditions comprising means for monitoring temperature, air relative humidity, soil relative humidity at the plant bed, concentration levels of carbon dioxide, radiation, irrigation scheduling, drainage, ventilation opening percentage and shading screen, the means for monitoring environmental conditions configured to transmit measurement data to the central unit for data processing and analysis of plant growth in the greenhouse.

54. The system of claim 53, wherein the means for monitoring temperature or air and soil relative humidity comprising a temperature or humidity measuring device placed in the vicinity of the weighing unit, the temperature or humidity device transmits its reading to the weighing unit through wire communication with a wire having sufficient length to allow the temperature or humidity device to stay in place as the weighing unit moves with the trellising system.

55. The system of claim 49, wherein the central unit further comprising means for displaying continuous distribution of the growth data in the greenhouse, possible reasons for their occurrence, statistical comparative analysis between different areas in the greenhouse, different species and treatments in different areas in the greenhouse, and suggesting strategies for improving plants growth to a user.

56. The system of claim 49, wherein said weighing units are detachable from said trellising system, said weighing units may be deployed in the greenhouse according to any pre-selected formation, said formation enabling collecting and analyzing data on plants growth from any particular area in the greenhouse.

57. The system of claim 49, further comprising an internet cloud enabling access to the central unit for retrieving data uploaded by remote computer databases to servers of the internet cloud, uploading data from the central unit accessible to the servers, the data accessible to remote computer databases, processors and monitoring and supervising means.

58. A method for monitoring plants in a greenhouse, the method comprising:
measuring the weight of a plant or group of plants from a top end of the plant or group of plants by a plurality of weighing units distributed in a greenhouse and trellising from an elevated wire between the wire and plants;
measuring values of climate and environmental conditions surrounding the plant or group of plants;
importing climate, environmental and any other growth- and growth-rate affecting conditions data surrounding the greenhouse;
recording the weight values measured in the weighing units;
recording the values of climate and environmental conditions;
transmitting the weight and climate and environmental values to a central unit through a communication network; and
storing, processing, analyzing the weight and climate and environmental values performing continuous dynamic growth simulation updated at regular time intervals and predicting plant growth at a central software in the central unit.

59. The method of claim 58, wherein the measuring of weight is made at selected time intervals between 10 and 30 minutes enabling continuous monitoring of plants weight.

60. The method of claim 58, wherein the weight values are measured with an error range of no more than ±2 grams for every 10 kg of plant weight.

61. The method of claim 58, further comprising calibrating scales of the weighing unit according to temperature, wherein a calibration curve produced for the calibrating scales is specific for each scale and independent of absolute temperature in the surrounding of the weighing unit.

62. The method of claim 58, further comprising identifying problems in the cultivating, treatment, process and control systems of a greenhouse and providing early and on-line indications on problems and irregularities in growth of the plants, said problems and irregularities comprising irregular irrigation cycles identified in the irrigation system, instabilities in temperature, air and soil humidity, concentration levels of carbon dioxide and radiation.

63. The method of claim 58, further comprising monitoring climate, environmental, labor and growth-affecting conditions in the greenhouse in the vicinity of plants weighed by the weighing units or at selected places in the greenhouse, wherein measured values of said conditions are processed according to specially formulated mathematical equations, the equations setting the ground for correlating between measured climate and environmental conditions and plants growth.

64. The method of claim 58, further comprising filtering background noise, identifying weighing events and identifying weighing units according to identification data, preferably the identification data comprising an identification serial number for spatial location of the weighing units.

65. The method of claim 58, further comprising extracting statistical models for strategy and decision-making for growth of plants in the greenhouse and optimal values of measured parameters and climate and environmental data.

66. The method of claim 58, further comprising providing spatial representation of plants performance and variation of the plants performance with time, the plants performance being expressed in diseases in the plants, growth rate and general response to development process of the plants.

67. A decision support system for operating and managing a greenhouse comprising means for collecting, recording, storing and analyzing plant-related and climate, environmental, labor and growth affecting conditions data, weighting and processing the data, providing comprehensive status of the greenhouse based on continuously updated database, and optimizing yield and economic growth of plants, wherein the means comprising a plurality of weighing units distributed over the greenhouse and wireless communication network comprising repeater-repeater means distributed in the greenhouse and central transmission means located in the greenhouse, the weighing units communicating wirelessly and continuously monitoring communication with the repeater-repeater means, the weighing units and communicating directly with the central transmission means upon failure of communication with the repeater-repeater means, wherein the communication between the weighing units and the repeater means or central transmission means enabling synchronizing weighing events taken at selected time intervals by the weighing units, the plurality of weighing units and wireless communication means enabling the formation of a moving kernel, the moving kernel providing a dynamic image of growth rate of plants in the greenhouse, the image varying with time, wherein a central unit communicating with the central transmission means comprises time-domain filters, spatial-domain filters, smoothing filters and edge-enhancement filters, and wherein the system is configured to predict daily plant growth based on plant growth affecting conditions data collected on last days preceding the day of predicting plant growth.

68. The decision support system of claims 67, wherein the time-domain filters and smoothing filters enable smoothing irregularities and highlighting non-linear events associated with the growth rate of plants, the irregularities comprising picking, deleafing, trimming and lowering, the events comprising variations in climate and environmental conditions in the greenhouse.

69. The decision support system of claim 67, wherein the spatial-domain filters, edge-enhancement filters and smoothing filters enable smoothing and highlighting location-dependent phenomena and defining areas boundaries in the greenhouse according to the phenomena, the location-dependent phenomena comprising local development of plant performance expressed in diseases in the plants, growth rate and general response of the plants, working-boundaries of a worker, areas of non-uniform circulation of humidity, variations in temperature, distribution of pesticides and non-uniformities in plants fertilization.

70. The decision support system of claim 69 further comprising means for correlating between present growth rate and past values of growth-influencing parameters, the means enabling such correlation lacking any in-advance information of the phenomena generating values of the growth-influencing parameters.

71. The decision support system of claim 67 further comprising means for deriving conclusions on the greenhouse operation, the means comprising statistical means for calculating growth rate data in plants in the greenhouse, wherein the means for deriving conclusions on the greenhouse operation enable producing and optimizing economic and cost-effective models of growth of plants in the greenhouse based on accumulated, historical and statistical data and quantifying growth strategy for analysis and decision-making activities.

72. The decision support system of claim 71, wherein the growth strategy comprises optimizing plants growth-rate affecting conditions, the growth-rate affecting conditions comprising fertilization, irrigation and water balance, heating, carbon dioxide (CO₂) concentration levels, application of ventilation, screen opening and closing and plant treatment regimens, the plant treatment regimens comprising plants lowering, deleafing, trimming, picking, harvesting and spraying and labor.

73. The decision support system of claim 72, wherein the growth strategy enables a user to maximize profits.
